(19) United States
(12) Patent Application Publication
(10) Pub. No.: US 2024/0189133 A1
(43) Pub. Date: Jun. 13, 2024

(54) AUXILIARY FRAME

(71) Applicant: InnoCare Optoelectronics Corporation, Tainan City (TW)

(72) Inventor: Shao-Han Li, Tainan City (TW)

(73) Assignee: InnoCare Optoelectronics Corporation, Tainan City (TW)

(21) Appl. No.: 18/502,100

(22) Filed: Nov. 6, 2023

(30) Foreign Application Priority Data
Dec. 13, 2022 (TW) 111147722

(51) Int. Cl. A61F 5/37

(52) U.S. Cl. CPC A61F 5/37

(57) ABSTRACT

An auxiliary frame for accommodating a flat panel detector is provided. A first side of the auxiliary frame includes two first fixing structures. The two first fixing structures are adjacent to a first edge of the flat panel detector and are respectively located on two sides of a first center axis of the flat panel detector. The first edge of the flat panel detector has a first length. A distance between the two first fixing structures is greater than or equal to half of the first length and less than the first length.

### CROSS-REFERENCE TO RELATED APPLICATION

This application claims the priority benefit of Taiwan application serial no. 111147722, filed on Dec. 13, 2022. The entirety of the above-mentioned patent application is hereby incorporated by reference herein and made a part of this specification.

### BACKGROUND

### Technical Field

The disclosure relates to an auxiliary frame, and in particular relates to an auxiliary frame for accommodating a flat panel detector.

### Description of Related Art

When shooting X-rays, based on the consideration of image quality, it is necessary to fix the position of the affected part or the patient. When encountering patients who cannot cooperate, such as patients with bedridden contractures, restless patients, children, or newborns, etc., tapes, bandages and other fixtures are usually used to fix the affected part or patients. However, there are many problems with the existing fixtures or fixing methods, such as poor fixing force, few fixing places, insufficient adjustment angles, prone to ghosting, or inability to be used with existing medical facilities.

### SUMMARY

The disclosure provides an auxiliary frame, which can assist in fixing the affected part or the patient, and improve the convenience of photo shooting or the image quality.

In an embodiment of the disclosure, the auxiliary frame is used for accommodating a flat panel sensor. A first side of the auxiliary frame includes two first fixing structures. The two first fixing structures are adjacent to a first edge of the flat panel sensor and are respectively located on two sides of a first center axis of the flat panel sensor. The first edge of the flat panel sensor has a first length. A distance between the two first fixing structures is greater than or equal to half of the first length and less than the first length.

In order to make the aforementioned features and advantages of the disclosure comprehensible, embodiments accompanied with drawings are described in detail below.

### BRIEF DESCRIPTION OF THE DRAWINGS

FIG. 1 and FIG. 2 are top schematic views of the auxiliary frame according to the first embodiment of the disclosure, respectively showing before and after the combination of the auxiliary frame and the fixing belt.

FIG. 3 is a side schematic view of the auxiliary frame according to the first embodiment of the disclosure.

FIG. 4A and FIG. 4B are cross-sectional schematic views of the auxiliary frame according to the second embodiment of the disclosure, respectively showing the cross-sections corresponding to the section line I-I′ and the section line II-II′ in FIG. 1.

FIG. 5A and FIG. 5B are bottom schematic views of the auxiliary frame according to the third embodiment of the disclosure, respectively showing the states when the fixing structures are opened and closed.

FIG. 6A and FIG. 6B are side schematic views of the auxiliary frame according to the fourth embodiment of the disclosure under different viewing angles, respectively showing the states when the fixing belts are stored and expanded.

FIG. 7 is a side schematic view of the auxiliary frame according to the fifth embodiment of the disclosure.

### DETAILED DESCRIPTION OF DISCLOSED EMBODIMENTS

References of the exemplary embodiments of the disclosure are to be made in detail. Examples of the exemplary embodiments are illustrated in the drawings. If applicable, the same reference numerals in the drawings and the descriptions indicate the same or similar parts.

Certain terms may be used throughout the disclosure and the appended patent claims to refer to specific elements. It should be understood by those of ordinary skill in the art that electronic device manufacturers may refer to the same element by different names. The disclosure does not intend to distinguish between elements that have the same function but have different names. In the following description and patent claims, words such as “comprising” and “including” are open-ended words, so they should be interpreted as meaning “including but not limited to . . . ”.

In the disclosure, wordings used to indicate directions, such as “up,” “down,” “front,” “back,” “left,” and “right,” merely refer to directions in the accompanying drawings. Therefore, the directional wordings are used to illustrate rather than limit the disclosure. In the accompanying drawings, the drawings illustrate the general features of the methods, structures, and/or materials used in the particular embodiments. However, the drawings shall not be interpreted as defining or limiting the scope or nature covered by the embodiments. For example, the relative sizes, thicknesses, and locations of the layers, regions, and/or structures may be reduced or enlarged for clarity.

A structure (or layer, element, substrate) described in this disclosure is located on/above another structure (or layer, element, substrate), may indicate that the two structures are adjacent and directly connected, or may indicate that the two structures are adjacent but not directly connected. Indirect connection means that there is at least one intermediate structure (or intermediate layer, intermediate element, intermediate substrate, intermediate spacer) between the two structures, the lower surface of one structure is adjacent to or directly connected to the upper surface of the intermediate structure, and the upper surface of another structure is adjacent to or directly connected to the lower surface of the intermediate structure. The intermediate structure can be composed of a single-layer or multi-layer physical structure or a non-physical structure, which is not limited thereto. In the disclosure, when a certain structure is disposed “on” other structures, it may mean that a certain structure is “directly” on other structures, or it means that a certain structure is “indirectly” on other structures, that is, at least one structure is sandwiched between a certain structure and other structures.

The terms “about”, “equal to”, “equal” or “same”, “substantially” or “generally” are interpreted as within 20% of a given value or range, or interpreted as within 10%, 5%, 3%, 2%, 1%, or 0.5% of the given value or range. Furthermore, the terms “range from a first value to a second value”, “range between a first value to a second value” mean that the range includes the first value, the second value, and other values in between.

The terms such as “first”, “second”, etc. used in the description and the patent claims are used to modify elements, which do not imply and represent that the (or these) elements have any previous ordinal numbers, and also does not represent the order of a certain element and another element, or the order of the manufacturing method. The use of these ordinal numbers is to only clearly distinguish an element with a certain name from another element with the same name. The same terms may not be used in the patent claims and the description, and accordingly, the first component in the description may be the second component in the patent claims.

In some embodiments of the disclosure, terms related to joining and connecting, such as “connected”, “interconnected”, etc., unless otherwise defined, may mean that two structures are in direct contact, or may also mean that two structures are not in direct contact, in which there are other structures located between these two structures. The terms related to joining and connecting can also include the case where both structures are movable, or both structures are fixed. Furthermore, the term “coupled” includes any direct and indirect means of electrical connection. Furthermore, the term “connected” includes a means of signal communication by which two elements or devices can directly or indirectly receive and/or transmit wireless signals.

The electrical connection or coupling described in the disclosure can refer to direct connection or indirect connection. In the case of a direct connection, the end points of two elements on a circuit directly connect to each other, or connect to each other through a conductive wire. In the case of indirect connection, a switch, a diode, a capacitor, an inductor, a resistor, other suitable elements, or a combination thereof, but not limited therein, is between the end points of two elements on a circuit.

In the disclosure, the thickness, length, and width may be measured by adopting a measurement method such as an optical microscope (OM), and the thickness or width can be measured from a cross-sectional image in an electronic microscope, but not limited thereto. In addition, any two values or directions used for comparison may have certain errors. Furthermore, the terms “a given range is from a first value to a second value”, “a given range is within a range from the first value to the second value”, or “a given range is between the first value to the second value” means that the given range includes the first value, the second value, and other values in between. If a first direction is perpendicular to a second direction, an angle between the first direction and the second direction may be between 80 degrees and 100 degrees; if the first direction is parallel to the second direction, an angle between the first direction and the second direction may be between 0 degrees and 10 degrees.

It should be noted that, in the following embodiments, the features in several different embodiments can be replaced, reorganized, and mixed to complete other embodiments without departing from the spirit of the disclosure. As long as the features of the various embodiments do not violate the spirit of the disclosure or conflict with one another, they can be mixed and matched arbitrarily.

Unless otherwise defined, all terms (including technical and scientific terms) used herein have the same meaning as commonly understood by one of ordinary skill in the art to which this disclosure belongs. It is understood that these terms, such as those defined in commonly used dictionaries, should be interpreted as having meanings consistent with the relevant art and the background or context of the disclosure, and should not be interpreted in an idealized or overly formal manner, unless otherwise defined in the embodiments of the disclosure.

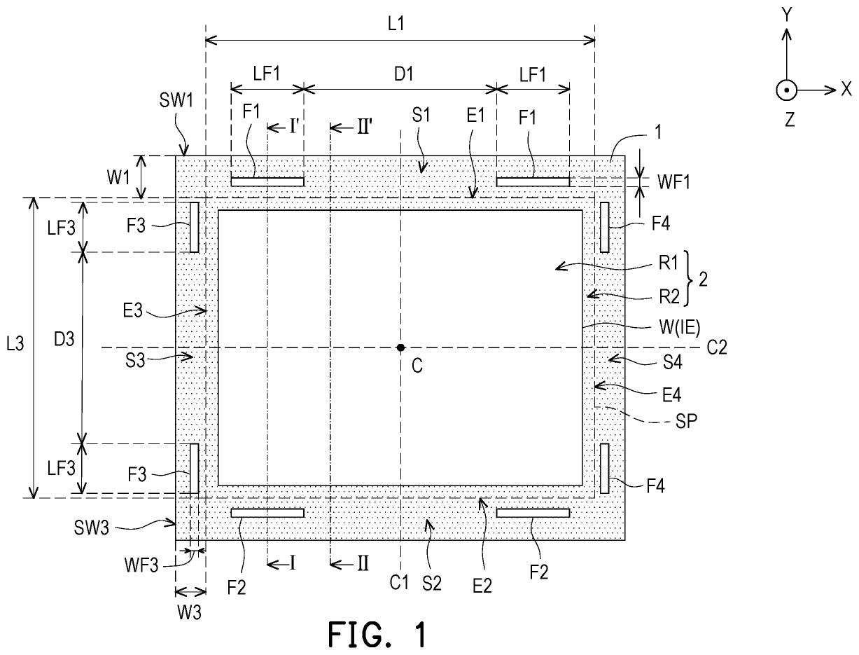

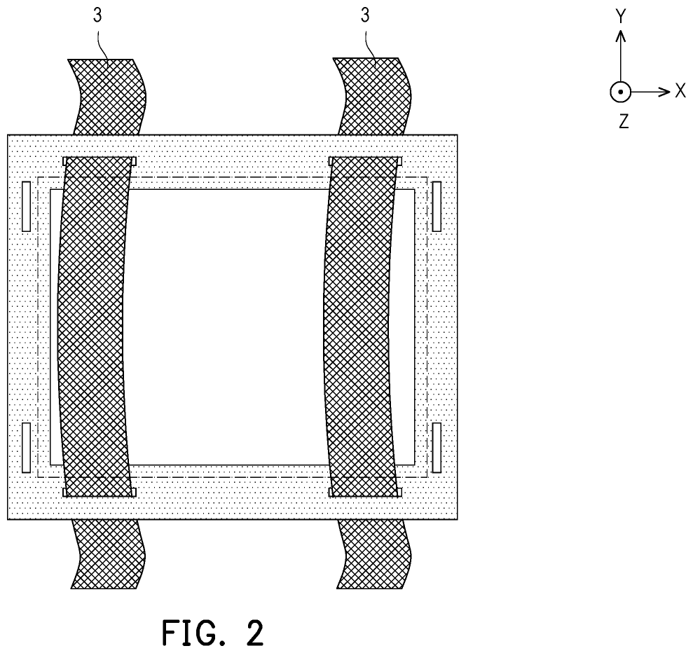

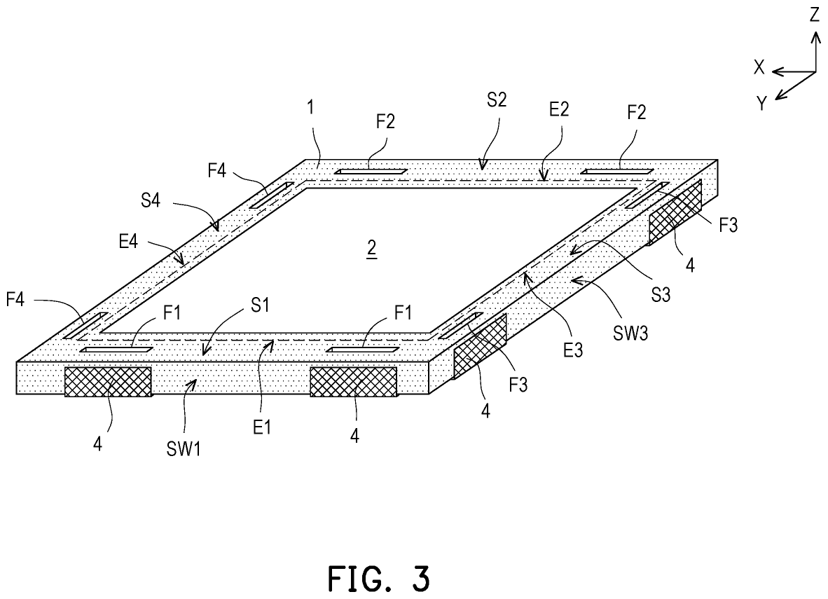

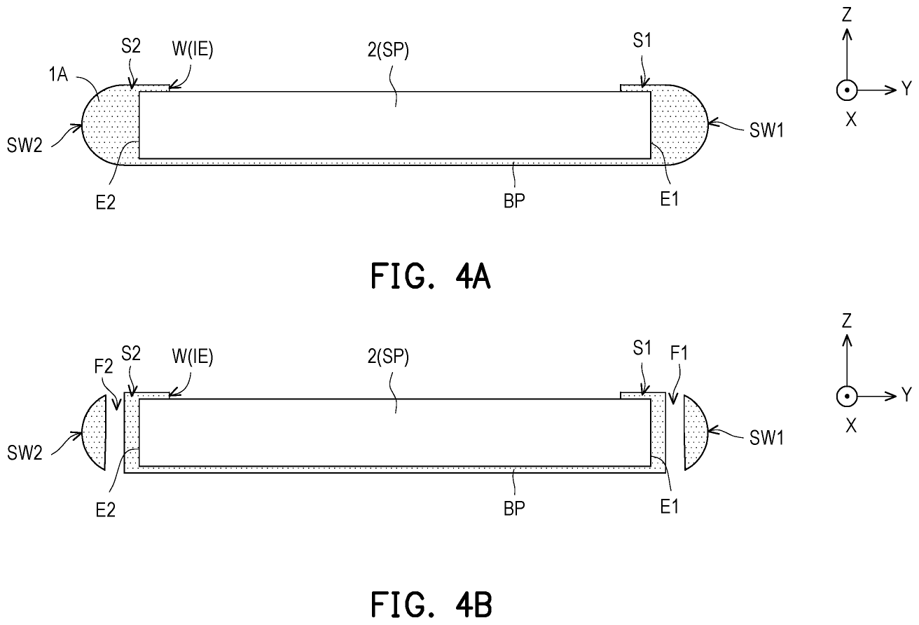

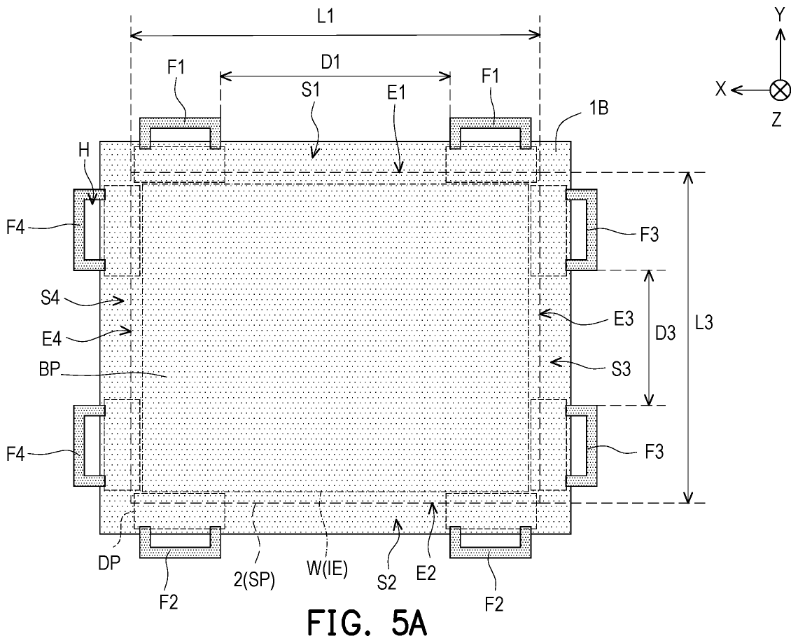

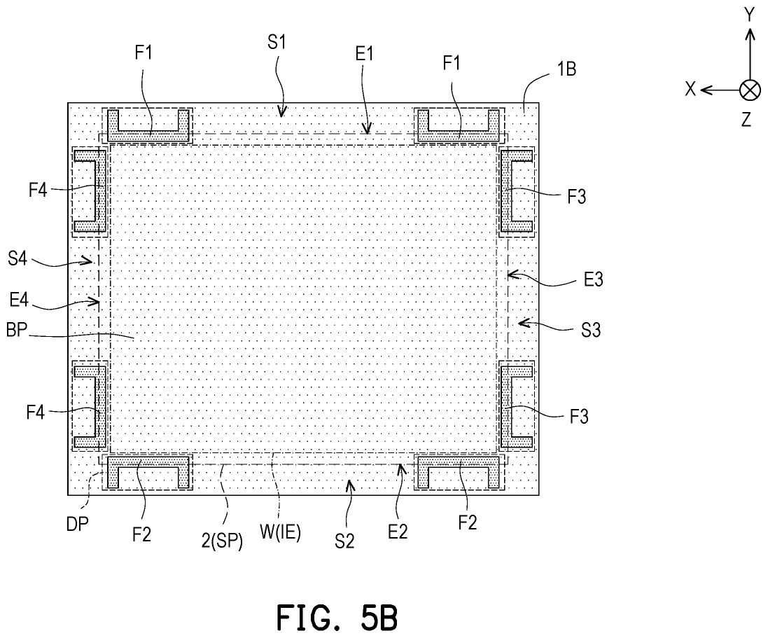

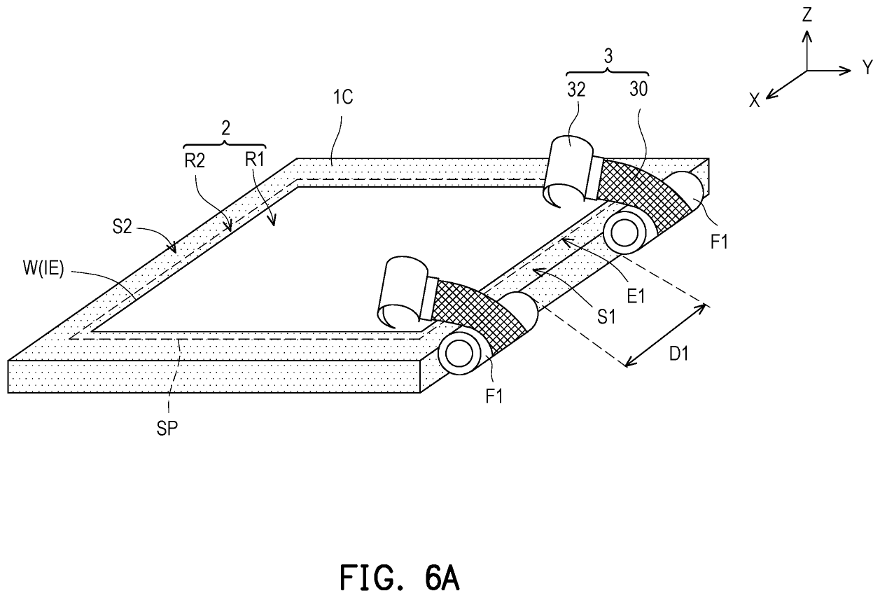

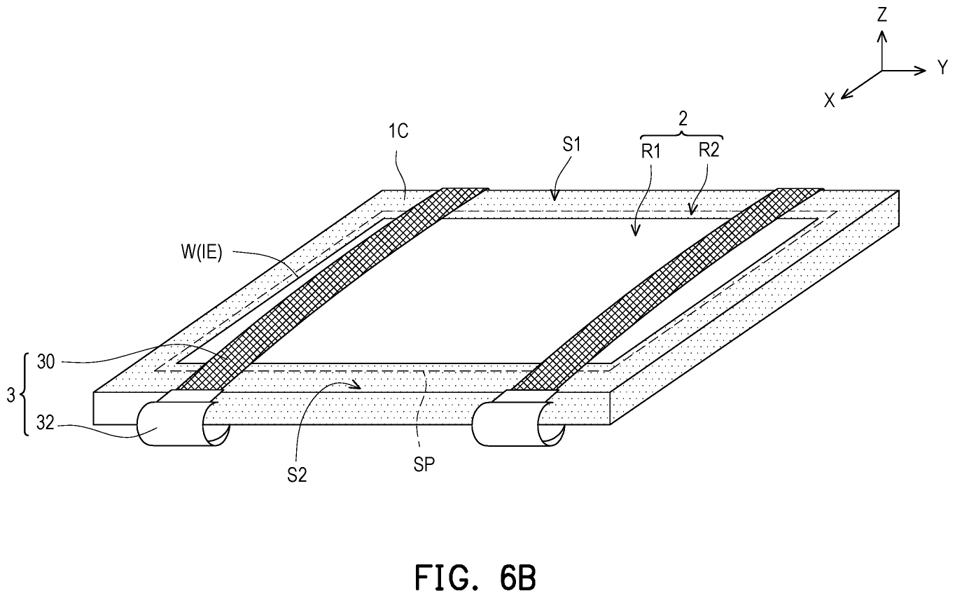

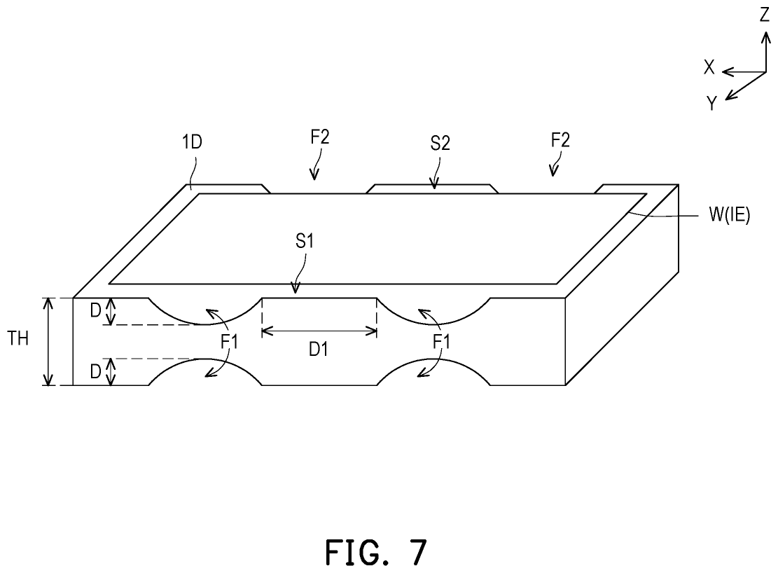

FIG. 1 and FIG. 2 are top schematic views of the auxiliary frame according to the first embodiment of the disclosure, respectively showing before and after the combination of the auxiliary frame and the fixing belt. FIG. 3 is a side schematic view of the auxiliary frame according to the first embodiment of the disclosure. FIG. 4A and FIG. 4B are cross-sectional schematic views of the auxiliary frame according to the second embodiment of the disclosure, respectively showing the cross-sections corresponding to the section line I-I′ and the section line II-II′ in FIG. 1. FIG. 5A and FIG. 5B are bottom schematic views of the auxiliary frame according to the third embodiment of the disclosure, respectively showing the states when the fixing structures are opened and closed. FIG. 6A and FIG. 6B are side schematic views of the auxiliary frame according to the fourth embodiment of the disclosure under different viewing angles, respectively showing the states when the fixing belts are stored and expanded. FIG. 7 is a side schematic view of the auxiliary frame according to the fifth embodiment of the disclosure.

Referring to FIG. 1, the auxiliary frame 1 can be used to accommodate a flat panel sensor 2. The flat panel sensor 2 is, for example, a panel-shaped mobile X-ray sensor, but not limited thereto. The auxiliary frame 1 may have an accommodating space SP. The accommodating space SP is, for example, a hollow region located inside the auxiliary frame 1, and the flat panel sensor 2 can be disposed in the accommodating space SP. The auxiliary frame 1 may also have a contact window W. The contact window W is, for example, an opening that connects the accommodating space SP with the outside of the auxiliary frame 1, and the edge of the contact window W is defined by the inner edge IE of the auxiliary frame 1. The contact window W can at least expose the sensing region R1 of the panel sensor 2, so that the panel sensor 2 can sense external light. Under this structure, at least the front portion of the auxiliary frame (the portion adjacent to the contact window W) can be made of a material with a low covering rate to light (e.g., X-ray), but not limited thereto. When the flat panel sensor 2 covers the sensing region R1, at least the front portion of the auxiliary frame 1 can be made of a material that can be penetrated by light (e.g., X-ray), so as to reduce the occurrence of ghosting. Materials that can be penetrated by X-ray may include, but are not limited to, acrylic or carbon fiber.

In some embodiments, the top view area of the contact window W may be designed to be smaller than the top view area of the accommodating space SP. Alternatively, the inner edge IE of the auxiliary frame 1 can be designed to overlap the panel sensor 2, for example, the peripheral region R2 of the panel sensor 2 can be covered by the auxiliary frame 1, thereby strengthening the fixing of the auxiliary frame 1 to the panel sensor 2 or reducing the relative displacement between the panel sensor 2 and the auxiliary frame 1, but not limited thereto.

The flat panel sensor 2 includes a first center axis C1 passing through the center C of the flat panel sensor 2 and perpendicular to the extending direction (direction X) of the first edge E1. The first side S1 of the auxiliary frame 1 may include two first fixing structures F1. The two first fixing structures F1 are adjacent to the first edge E1 of the panel sensor 2 and are respectively located on opposite sides of the center C of the panel sensor 2 (e.g., the left and right sides in the top view), that is, the two first fixing structures F1 are respectively located on two sides of the first center axis C1. The first edge E1 of the flat panel sensor 2 has a first length L1. The distance D1 between the two first fixing structures F1 may be greater than or equal to half of the first length L1 and less than the first length L1.

In some embodiments, as shown in FIG. 1 and FIG. 2, the first fixing structure F1 may be a through hole penetrating through the first side S1 of the auxiliary frame 1 in the direction Z. The two first fixing structures F1 may be arranged in the direction X and extend in the direction X. The distance D1 between the two first fixing structures F1 is, for example, the minimum distance between the two first fixing structures F1 in the direction X. The fixing belt 3 can be combined with the auxiliary frame 1 through the first fixing structure F1. For example, one end of the fixing belt 3 can pass through the first fixing structure F1 to assist in fixing the patient or the affected part. The fixing belt 3 can be an elastic belt, an elastic cord, a contracting belt, or a restraint belt, but not limited thereto. Furthermore, the fixing belt 3 may be penetrated by X-rays. Through the design that the distance D1 between the two first fixing structures F1 is greater than or equal to half of the first length L1 and less than the first length L1, the covering of the center of the sensing region R1 by the fixing belt 3 can be reduced, thereby reducing the influence of the fixing belt 3 on the image quality (e.g., ghosting) when shooting X-rays.

In some embodiments, the ratio of the long edge length of the fixing structure to the length of the corresponding edge of the flat panel sensor 2 can be designed to improve the convenience and flexibility when used with the fixing belt or improve the quality of the captured image. For example, when the top view shape of the first fixing structure F1 is a rectangle, the long edge length LF1 of the rectangle can be designed to be less than or equal to a quarter of the first length L1, but not limited thereto.

In some embodiments, the ratio of the short edge width of the fixing structure to the physical portion width of the corresponding side of the auxiliary frame 1 can also be designed to improve the structural strength of the auxiliary frame 1. For example, the ratio of the short edge width WF1 of the first fixing structure F1 to the physical portion width W1 of the first side S1 (e.g., the distance between the first side wall surface SW1 and the first edge E1 of the flat panel sensor 2 in the direction Y) may fall between ¼ and ½, so as to reduce the probability of breaking the first side S1, but not limited thereto. In some embodiments, the physical portion width W1 is, for example, between 1.5 cm and 2 cm, that is, 1.5 cm≤W1≤2 cm, and the short edge width WF1 is, for example, between 0.5 cm and 1 cm, that is, 0.5 cm≤WF1≤1 cm, but not limited thereto.

In some embodiments, by increasing the number of fixing structures or the relative disposition relationship of multiple fixing structures, it improves the fixing force, the fixing position, or the adjustable angle when fixing the patient or the affected part, improves the convenience and flexibility when used with the fixing belt, improves the image quality of the photo shooting, or reduces the movement of the patient or the affected part during photo shooting.

For example, the second side S2 of the auxiliary frame 1 may include two second fixing structures F2. The second side S2 and the first side S1 are opposite sides of the auxiliary frame 1 (e.g., upper and lower sides in a top view). The two second fixing structures F2 are adjacent to the second edge E2 of the panel sensor 2 and are respectively located on opposite sides of the center C of the panel sensor 2 (e.g., the left and right sides in the top view), that is, the two second fixing structures F2 are respectively located on two sides of the first center axis C1. For example, the second edge E2 of the flat panel sensor 2 also has the first length L1. The distance between the two second fixing structures F2 (not marked, refer to the distance D1) can be designed to be greater than or equal to half of the first length L1 and less than the first length L1, to reduce the covering of the center of the sensing region R1 by the fixing belt 3, thereby reducing the influence of the fixing belt 3 on the image quality when shooting X-rays.

For the related design of the second fixing structure F2 (e.g., structure, arrangement direction, extending direction, top view shape, length, or width, etc.), refer to the relevant description of the first fixing structure F1, which are not repeated herein.

In some embodiments, the flat panel sensor 2 further includes a second center axis C2 passing through the center C of the flat panel sensor 2 and perpendicular to the extending direction (direction Y) of the third edge E3. The third side S3 of the auxiliary frame 1 may include two third fixing structures F3. The third side S3 is connected between the second side S2 and the first side S1. The two third fixing structures F3 are adjacent to the third edge E3 of the panel sensor 2 and are respectively located on opposite sides of the center C of the panel sensor 2 (e.g., the upper and lower sides in the top view), that is, the two third fixing structures F3 are respectively located on two sides of the second center axis C2. The third edge E3 of the flat panel sensor 2 has a third length L3. The distance D3 between the two third fixing structures F3 can be designed to be greater than or equal to half of the third length L3 and less than the third length L3.

In some embodiments, the third fixing structure F3 may be a through hole penetrating through the third side S3 of the auxiliary frame 1 in the direction Z. The two third fixing structures F3 may be arranged in the direction Y and extend in the direction Y. Although not shown, the fixing belt can be combined with the auxiliary frame 1 through the third fixing structure F3. For example, one end of the fixing belt can pass through the third fixing structure F3 to assist in fixing the patient or the affected part. Through the design that the distance D3 between the two third fixing structures F3 is greater than or equal to half of the third length L3 and less than the third length L3, the covering of the center of the sensing region R1 by the fixing belt can be reduced, thereby reducing the influence of the fixing belt on the image quality when shooting X-rays.

In some embodiments, the top view shape of the third fixing structure F3 is a rectangle, and the long edge length LF3 of the rectangle can be designed to be less than or equal to a quarter of the third length L3 to improve the convenience and flexibility when used with the fixing belt or improve the quality of the captured image.

In some embodiments, the ration of the short edge width WF3 of the third fixing structure F3 to the physical portion width W3 of the third side S3 (e.g., the distance between the third side wall surface SW3 and the third edge E3 of the flat panel sensor 2 in the direction X) can fall between ¼ and ½, so as to reduce the probability of breaking the third side S3, but not limited thereto. In some embodiments, the physical portion width W3 is, for example, between 1.5 cm and 2 cm, that is, 1.5 cm≤W3≤2 cm, and the short edge width WF3 is, for example, between 0.5 cm and 1 cm, that is, 0.5 cm≤ WF3≤1 cm, but not limited thereto.

In some embodiments, the fourth side S4 of the auxiliary frame 1 may include two fourth fixing structures F4. The fourth side S4 is connected between the second side S2 and the first side S1, and the fourth side S4 and the third side S3 are opposite sides of the auxiliary frame 1 (e.g., left and right sides in a top view). The two fourth fixing structures F4 are adjacent to the fourth edge E4 of the panel sensor 2 and are respectively located on opposite sides of the center C of the panel sensor 2 (e.g., the upper and lower sides in the top view), that is, the two fourth fixing structures F4 are respectively located on two sides of the second center axis C2. For example, the fourth edge E4 of the flat panel sensor 2 also has a third length L3. The distance between the two fourth fixing structures F4 (not marked, refer to the distance D3) can be designed to be greater than or equal to half of the third length L3 and less than the third length L3, to reduce the covering of the center of the sensing region R1 by the fixing belt (not shown in FIG. 2), thereby reducing the influence of the fixing belt on the image quality when shooting X-rays.

For the related design of the fourth fixing structure F4 (e.g., structure, arrangement direction, extending direction, top view shape, length, or width, etc.), refer to the relevant description of the third fixing structure F3, which are not repeated herein.

In some embodiments, as shown in FIG. 3, the first side wall surface SW1 of the auxiliary frame 1 is adjacent to the first edge E1 of the panel sensor 2, and an adhesive fixing element 4 may be disposed on the first side wall surface SW1, so that the fixing belt 3 (refer to FIG. 2) passing through the first fixing structure F1 can be adhesively fixed. The adhesive fixing element 4 may comprise Velcro or other elements with adhesion to the fixing belt 3. In some embodiments, the adhesive fixing element 4 can further extend to the bottom surface of the auxiliary frame 1 (not shown in FIG. 3).

In some embodiments, the number of adhesive fixing elements 4 can be multiple, and the multiple adhesive fixing elements 4 can be disposed corresponding to multiple fixing structures (e.g., the first fixing structure F1, the second fixing structure F2, the third fixing structure F3, and the fourth fixing structure F4), that is, the disposition relationship between the adhesive fixing element 4 and the fixing structure may be one-to-one, but not limited thereto. In other implementations, although not shown, the disposition relationship between the adhesive fixing element and the fixing structure can be one-to-many, for example, one adhesive fixing element (not shown) that can cover a larger area replaces two adhesive fixing elements 4 corresponding to the two first fixing structures F1, the relative disposition relationship between other fixing structures and the adhesive fixing elements can be deduced in the same way, and are not repeated herein.

In some embodiments, although not shown in FIG. 3, one end of the fixing belt can pass through the first fixing structure F1 and be adhesively fixed through the adhesive fixing element 4 disposed corresponding to the first fixing structure F1, and the other end of the fixing belt can pass through the second fixing structure F2 and be adhesively fixed through the adhesive fixing element 4 disposed corresponding to the second fixing structure F2, but not limited thereto. For example, the other end of the fixing belt can only be adhesively fixed through the adhesive fixing element 4 disposed corresponding to the second fixing structure F2 without passing through the second fixing structure F2. Moreover, in addition to passing through two opposite fixing structures, the fixing belt can also pass through two adjacent fixing structures, such as passing through two first fixing structures F1 or passing through the first fixing structure F1 and the adjacent fourth fixing structure F4, but not limited thereto.

In other embodiments, although not shown in FIG. 3, the auxiliary frame 1 may not include the adhesive fixing element 4, and the fixing belt passing through the fixing structure may be fixed on one side of the auxiliary frame 1 through knotting. Alternatively, when there is a U-shaped hook or a J-shaped hook at one end of the fixing belt, the U-shaped hook or the J-shaped hook can be directly buckled to the opposite frame without fixing through knotting or the adhesive fixing element 4.

In this embodiment, the auxiliary frame can be used with existing medical facilities. For example, when encountering a newborn patient, the auxiliary frame 1 accommodating the flat panel sensor 2 can be placed between the newborn patient and the newborn bed or incubator, and the newborn patient is fixed through at least one fixing structure and a fixing belt to facilitate X-ray shooting. On the other hand, encountering patients who cannot cooperate with X-ray shooting, such as patients with bedridden contractures, restless patients, patients with weak hands and feet, deformed hands and feet, or children, the auxiliary frame 1 accommodating the flat panel sensor 2 can be moved according to the requirement of the affected part or the patient. For example, the auxiliary frame 1 accommodating the flat panel sensor 2 can be placed under the patient or the affected part (e.g., the patella, palm, chest cavity, abdomen, limbs, head, or neck) to shoot X-rays of the patient or affected part. In addition, the patient or the affected part can be fixed through multiple fixing structures and at least one fixing belt, thereby improving the image quality of the photo shooting. Through the design of the number of the fixing structures and the relative disposition positions, it facilitates improving the fixing force, the fixing position, or the adjustable angle when fixing the patient or the affected part, improving the convenience and flexibility when used with the fixing belt, improving the image quality of the photo shooting, or reducing the movement of the patient or the affected part during photo shooting.

Referring to FIG. 4A and FIG. 4B, the main difference between the auxiliary frame 1A and the auxiliary frame 1 in FIG. 3 is that the side wall surfaces of the auxiliary frame 1A (including the first side wall surface SW1 and the second side wall surface SW2) are arc surfaces. Through the design of the arc surface, the feeling of a foreign body or discomfort when the patient or the affected part is in contact with the auxiliary frame 1A can be reduced.

In some embodiments, the backside portion BP of the auxiliary frame 1A opposite to the contact window W may be made of low-scattering materials to reduce the interference of backscattered light on image quality. For example, the low-scattering material may include aluminum, magnesium, aluminum alloy, or magnesium alloy, but not limited thereto. Any embodiment of the disclosure can be changed accordingly, and are not repeated below.

In some embodiments, although not shown in FIG. 4A and FIG. 4B, an adhesive fixing elements 4 (refer to FIG. 3) may be disposed on the side wall surfaces of the auxiliary frame 1A (including the first side wall surface SW1 and the second side wall surface SW2). In some embodiments, the adhesive fixing element 4 may further extend to the bottom surface of the auxiliary frame 1 (e.g., under the backside portion BP).

Referring to FIG. 5A and FIG. 5B, the main differences between the auxiliary frame 1B and the auxiliary frame 1 in FIG. 3 are described as follows. In the auxiliary frame 1B, the fixing structure (including the first fixing structure F1, the second fixing structure F2, the third fixing structure F3, and the fourth fixing structure F4) is a buckle. When shooting, the fixing structure can be adjusted to an opened state so that the fixing belt can pass through the hole H of the buckle to fix the patient or affected part; and when the shooting is over, the fixing structure can be adjusted to a closed state for easy storage.

In some embodiments, the fixing structure may be disposed on the back side (back side part BP) of the auxiliary frame 1B, and the back side part BP may include a plurality of recesses DP respectively corresponding to the plurality of fixing structures. When the fixing structures are closed, each fixing structure can be stored in a corresponding recess DP, so that the back surface of the auxiliary frame 1B is flat and easy to store, but not limited thereto. In other embodiments, although not shown, the fixing structure may be disposed on the front surface or the side wall surfaces of the auxiliary frame 1B and located outside the sensing region R1 (refer to FIG. 1).

In some embodiments, although not shown in FIG. 5A and FIG. 5B, the side wall surfaces of the auxiliary frame 1B can also be designed with an arc surface, so as to reduce the feeling of a foreign body or discomfort when the patient or the affected part is in contact with the auxiliary frame 1B.

In some embodiments, although not shown in FIG. 5A and FIG. 5B, the auxiliary frame 1B can be the casing of the flat panel sensor 2, and the aforementioned buckle (including the first fixing structure F1, the second fixing structure F2, the third fixing structure F3, and the fourth fixing structure F4) may be disposed on the casing of the flat panel sensor 2 and located outside the sensing region R1 (refer to FIG. 1).

Referring to FIG. 6A and FIG. 6B, the main differences between the auxiliary frame 1C and the auxiliary frame 1 in FIG. 1 are described as follows. In the auxiliary frame 1C, the first fixing structure F1 is, for example, a seat belt connector. The fixing belt 3 includes an elastic belt 30 and a hook member (such as a U-shaped hook or a J-shaped hook) 32, in which the elastic belt 30 is connected between the first fixing structure F1 and the hook member 32. When shooting, as shown in FIG. 6B, the elastic belt 30 can be stretched to buckle the hook member 32 to the second side S2, thereby fixing the patient or the affected part; and when the shooting is over, as shown in FIG. 6A, the hook member 32 can be separated from the second side S2, so that the elastic belt 30 can be rolled back to the seat belt connector (the first fixing structure F1).

In some embodiments, although not shown in FIG. 6A and FIG. 6B, the side wall surfaces of the auxiliary frame 1C can also be designed with an arc surface, so as to reduce the feeling of a foreign body or discomfort when the patient or the affected part is in contact with the auxiliary frame 1C.

Referring to FIG. 7, the main differences between the auxiliary frame 1D and the auxiliary frame 1C in FIG. 6A are described as follows. In the auxiliary frame 1D, the fixing structures (including the first fixing structure F1 and the second fixing structure F2) are recessed, and the depth D of the recess is smaller than the thickness TH of the auxiliary frame 1D. In addition, the number of each of the first fixing structures F1 and the second fixing structures F2 is, for example, four, two of which are disposed adjacent to the front surface of the auxiliary frame 1D, and the remaining two are disposed on the back surface of the auxiliary frame 1D.

The recess allows the fixing belt (not shown in FIG. 7) to pass through without moving the auxiliary frame 1D, thus improving the convenience of fixing and dismounting.

To sum up, in the embodiments of the disclosure, the auxiliary frame can be used with existing medical facilities. When encountering a patient who cannot or is difficult to cooperate with when shooting X-rays, the auxiliary frame can be moved according to the requirement of the affected part or the patient. The patient or the affected part can be fixed through multiple fixing structures and at least one fixing belt, thereby improving the image quality of the photo shooting. Through the design of the number of the fixing structures and the relative disposition positions, it facilitates improving the fixing force, the fixing position, or the adjustable angle when fixing the patient or the affected part, improving the convenience and flexibility when used with the fixing belt, improving the image quality of the photo shooting, or reducing the movement of the patient or the affected part during photo shooting.

The foregoing embodiments are only used to illustrate the technical solutions of the disclosure, but not to limit the disclosure; although the disclosure has been described in detail with reference to the foregoing embodiments, persons of ordinary skill in the art should understand that the technical solutions described in the foregoing embodiments can still be modified, or parts or all of the technical features thereof can be equivalently replaced; however, these modifications or substitutions do not deviate the essence of the corresponding technical solutions from the scope of the technical solutions of the embodiments of the disclosure.

Although the embodiments of the disclosure and the advantages thereof have been disclosed above, it should be understood that any person with ordinary skill in the art can make changes, substitutions, and modifications without departing from the spirit and scope of the disclosure, and the features of the various embodiments can be arbitrarily mixed and replaced with one another to form other new embodiments. In addition, the protection scope of the disclosure is not limited to the processes, machines, manufactures, compositions of the matter, devices, methods, and steps in the specific embodiments described in the specification. It is understood that processes, machines, manufactures, compositions of matter, devices, methods, and steps developed in the present or in the future can be used in accordance with the disclosure as long as they can implement substantially the same functions or achieve substantially the same results in the embodiments described herein. Therefore, the protection scope of the disclosure includes the above-mentioned processes, machines, manufactures, compositions of matter, devices, methods, and steps. In addition, each of the claims constitutes a separate embodiment, and the protection scope of the disclosure also includes each of the combinations of the claims and the embodiments. The protection scope of the disclosure shall be determined by the appended patent claims.

What is claimed is:

1. An auxiliary frame, used for accommodating a flat panel sensor, wherein a first side of the auxiliary frame comprises two first fixing structures, the two first fixing structures are adjacent to a first edge of the flat panel sensor and are respectively located on two sides of a first center axis of the flat panel sensor, wherein the first edge of the flat panel sensor has a first length, a distance between the two first fixing structures is greater than or equal to half of the first length and less than the first length.

2. The auxiliary frame according to claim 1, wherein the first fixing structure is a through hole.

3. The auxiliary frame according to claim 1, wherein the first fixing structure is a recess, and a depth of the recess is smaller than a thickness of the auxiliary frame.

4. The auxiliary frame according to claim 1, wherein the first fixing structure is a buckle.

5. The auxiliary frame according to claim 1, wherein the first fixing structure is a seat belt connector.

6. The auxiliary frame according to claim 1, wherein a top view shape of the first fixing structure is a rectangle, and a long edge length of the rectangle is less than or equal to a quarter of the first length.

7. The auxiliary frame according to claim 6, wherein a ratio of a short edge width of the first fixing structure to a physical portion width of the first side falls between ¼ and ½.

8. The auxiliary frame according to claim 1, wherein a second side of the auxiliary frame comprises two second fixing structures, the second side and the first side are opposite sides of the auxiliary frame, the two second fixing structures are adjacent to a second edge of the flat panel sensor and are respectively located on two sides of the first center axis of the flat panel sensor, wherein the second edge has the first length, and a distance between the two second fixing structures is greater than or equal to half of the first length and less than the first length.

9. The auxiliary frame according to claim 8, wherein the second fixing structure is a through hole.

10. The auxiliary frame according to claim 8, wherein a top view shape of the second fixing structure is a rectangle, and a long edge length of the rectangle is less than or equal to a quarter of the first length.

11. The auxiliary frame according to claim 10, wherein a ratio of a short edge width of the second fixing structure to a physical portion width of the second side falls between ¼ and ½.

12. The auxiliary frame according to claim 8, wherein a third side of the auxiliary frame comprises two third fixing structures, the third side is connected between the second side and the first side, the two third fixing structures are adjacent to a third edge of the flat panel sensor and are respectively located on two sides of a second center axis of the flat panel sensor, wherein the third edge has a third length, and a distance between the two third fixing structures is greater than or equal to half of the third length and less than the third length.

13. The auxiliary frame according to claim 12, wherein the third fixing structure is a through hole.

14. The auxiliary frame according to claim 12, wherein a top view shape of the third fixing structure is a rectangle, and a long edge length of the rectangle is less than or equal to a quarter of the third length.

15. The auxiliary frame according to claim 14, wherein a ratio of a short edge width of the third fixing structure to a physical portion width of the third side falls between ¼ and ½.

16. The auxiliary frame according to claim 12, wherein a fourth side of the auxiliary frame comprises two fourth fixing structures, the fourth side and the third side are opposite sides of the auxiliary frame, the two fourth fixing structures are adjacent to a fourth edge of the flat panel sensor and are respectively located on two sides of the second center axis of the flat panel sensor, wherein the fourth edge has the third length, and a distance between the two fourth fixing structures is greater than or equal to half of the third length and less than the third length.

17. The auxiliary frame according to claim 1, wherein the auxiliary frame comprises X-ray penetrable material.

18. The auxiliary frame according to claim 1, wherein an inner edge of the auxiliary frame overlaps the flat panel sensor.

19. The auxiliary frame according to claim 1, wherein a first side wall surface of the auxiliary frame is adjacent to the first edge of the flat panel sensor, and an adhesive fixing element is disposed on the first side wall surface.

20. The auxiliary frame according to claim 1, wherein a first side wall surface of the auxiliary frame is adjacent to the first edge of the flat panel sensor, and the first side wall surface is an arc surface.
